(19) United States
(12) Patent Application Publication
(10) Pub. No.: US 2016/0022490 A1
(43) Pub. Date: Jan. 28, 2016

(54) DEVICE AND METHOD FOR PERFORMING THERMAL KERATOPLASTY USING HIGH INTENSITY FOCUSED ULTRASOUNDS

(71) Applicant: SABANCI UNIVERSITY, Tuzla - Istanbul (TR)

(72) Inventors: Arif Sanli Ergun, Cayyolu - Ankara (TR); Rupak Bardhan Roy, Tuzla - Instanbul (TR); Ayhan Bozkurt, Tuzla - Instanbul (TR)

(21) Appl. No.: 14/427,460

(22) PCT Filed: Nov. 12, 2013
(86) PCT No.: PCT/EP2013/073545

§ 371 Date: Mar. 11, 2015

(51) Int. Cl. A61F 9/007; A61B 8/08; A61B 8/00; A61B 8/14; A61B 8/10; A61N 7/02

(52) U.S. Cl. CPC A61F 9/00745; A61B 8/10; A61N 7/02; A61B 8/4281; A61B 8/4494; A61B 8/14; A61B 8/4455; A61B 8/5207; A61N 2007/0052

(57) ABSTRACT

A device for thermal keratoplasty, the device comprising a plurality of ultrasonic transducers for emitting ultrasound waves, wherein the ultrasound waves of at least one of the transducers is focused on a corresponding area of the cornea in order to heat these area and cause collagen shrinkage and at least one of the transducers is capable of receiving ultrasound waves for ocular imaging.

### 1. TECHNICAL FIELD

The present invention relates generally to the field of application of ultrasonic waves for ocular imaging and thermal keratoplasty. The present invention particularly relates to devices, systems and methods for performing thermal keratoplasty as well as intra-ocular imaging, to be used for the treatment of presbyopic astigmatism and hyperopia and even in some cases of irregular optical aberations by changing the shape of the cornea.

### 2. BACKGROUND OF THE INVENTION

Various methods for changing the corneal curvature have been developed through the last years to control and cure the ever increasing cases of myopia, hyperopia, astigmatism and other ocular vision irregularities.

In Radial Keratotomy (RK) radial incisions were performed on the cornea with a steel blade or diamond knife. The procedure was useful, but the radial incisions weaken the cornea and affect its integrity. Further such steel blade and diamond knife incisions can create ocular infections which may occur long after the surgeries are performed. Additionally the post-operative scars results in very bad refractive glares.

Keratomileusis is the surgical improvement of the refractive state of the cornea performed by lifting the front surface of the eye by forming a thin hinged flap under which the shape of the cornea is changed by using an excimer laser or other surgical device. Before the advent of the excimer laser, keratomileusis was performed using a cryolathe, which froze thin flaps of corneal tissue and lathe cut them much like one cuts the lens of a pair of glasses. After thawing, these reshaped flaps were placed under the front flap to correct visual improvement. The lathe cutting generates unwanted surgical complexities. Even such processes have poor predictability.

The document U.S. Pat. No. 4,840,175 describes LASIK or Laser-Assisted in situ Keratomileusis. In these methods the process involves creating a thin flap on the eye, folding it away to enable remodeling of the tissue beneath with a laser and repositioning the flap. This process has quite a few serious flap related problems like irregular flaps, flap striae and epithelial ingrowths. High amount of aberrations are common in this process after the ablation is finished.

In Photorefractive Keratectomy (PRK) the frontal corneal epithelium is removed mechanically and then a considerable amount of cornea is ablated to change the curvature. The most important problem of PRK lies in the fact that it produces huge post-surgical haze even years after the ablation procedure. An additional issue with the process is that the epithelium removed is not very smooth and the epithelium regrowth is not very homogeneous which may result in haze.

LASEK or LAser Epithelial Keratomileusis reduced this haze effect by using alcohol for softening the corneal epithelium and not cutting it mechanically, Post ablation, the epithelium is replaced in the previous position. The haze related problem was reduced, but this process produced huge post-operative pain. Additionally it is seen that even the use of optimally produced alcohol solution (18-20%) causes some toxic effect on the corneal tissues.

In Epi-LASIK a device mechanically removes the corneal epithelium producing a smooth and uniform epithelial flap which can be replaced post ablation. Epi-LASIK almost solved some of the problems of PRK and LASEK. Still, the surgery has quite a long healing procedure and most importantly patients with thin cornea cannot afford such a surgery.

Unlike the above mentioned procedures, Thermal Keratoplasty (TK) uses a noninvasive procedure where corneal curvature is produced by inducing heat into the corneal stroma and producing collagen shrinkage in a required pattern around the temperature of 60 to 65 degree Celsius approximately. This method generates the bulging out of the central corneal space reducing hyperopia. For treating Astigmatism a couple of spots facing each other on the cylindrical axis are heated to change the corneal shape, in the way as needed. A couple of TK processes has been developed, namely Laser Thermal Keratoplasty (LTK), and Conductive Keratoplasty (CK).

Among the two LTK is less invasive and advantageous, as CK has problems like initial over-correction, inducing astigmatism, scarring the cornea etc. In LTK two rings are used for fixing multiple LASER spots around the cornea. The number of spots is determined by the amount of shrinkage as needed, as more spots increases the belt effect producing more bulging of the central corneal zone. One problem that LTK has is the problem of regression, and thus there is need of redoing the process more than once. But, as LTK is being done only to patients above 40 years of age, the patients in question are mostly affected by Presbyopia. So, refractive correction is a frequent requirement and thus repeating LTK more than once is not a major problem. Thus it is seen that LTK is an advantageous way of treating presbyopic hyperopia and astigmatism. Even in some cases it can treat irregular ectasia, providing localized collagen shrinkage produced by a single spot LASER heating on the ectasia effected area.

Recent research has reported the development of a more sophisticated version of LTK called Optimal Keratoplasty (Opti-K). In such a system the unnecessary heating of the entire cornea is controlled by the use of a Sapphire corneal suction ring that acts as a heat sink reducing the unwanted heating of the whole cornea and keeping the heating process highly localized. However, with LTK or Opti-K it is not possible to perform A-scan ultrasonography and cornea correction in vivo with a single system integrated in single packaging.

A further problem arises with the LASER guided systems. Even though there are various ocular imaging techniques like Ultrasound A-scan, Ultrasound B-scan, Fundus Photography, Fluorescine Angiography, Optical Coherence Tomography, and/or other available processes, the imaging and surgery systems work separately, resulting in a complex and expensive system. Furthermore, operation time is long because thermal keratoplasty and ocular imaging are performed with independent systems. Cost is also high because it depends on the operation time.

The use of high intensity focused ultrasound (HIFU) for the treatment or therapy of human tissue is known in the art. For example the documents WO 2010/002646 A1, US 2008/0039724 A1 or U.S. Pat. No. 6,719,694 disclose devices with ultrasonic transducers for heating of human tissue and for imaging. However, these devices are designed for treatment of tumors, blood vessels, cancer and the like and are unsuitable for thermal keratoplasty.

The document U.S. Pat. No. 4,484,569 discloses a device for thermal ultrasonic treatment of the retina. This device uses a single focused ultrasonic transducer for the therapy and a separate ultrasonic transducer for imaging. Further, the device must be re-located in order to treat different areas of the retina.

The document U.S. Pat. No. 5,230,334 discloses a thermal keratoplasty device and method using a single ultrasound transducer. The transducer ultrasounds are transmitted through a waveguide and focused with a lens. The transmission and focusing system is therefore complex and expensive. Operation time with this device is long because the single transducer must be focused and moved around the area to be treated. Furthermore, ocular imaging must be performed with another device, further increasing operation time and system complexity.

It is therefore an object of the present invention to provide solutions to the above mentioned problems, especially to reduce system complexity and costs.

### 3. SUMMARY OF THE INVENTION

The above mentioned problem is solved by a device for thermal keratoplasty according claim 1, a system for both ocular imaging and thermal keratoplasty according claim 10 and a method for both ocular imaging and thermal keratoplasty according claim 11.

Particularly the above mentioned problem is solved by a device for thermal keratoplasty, the device comprising a plurality of ultrasonic transducers for emitting ultrasound waves, wherein the ultrasound waves of at least one of the transducers is focused on a corresponding area of the cornea in order to heat these area and cause collagen shrinkage, and at least one of the transducers is capable of receiving ultrasound waves for ocular imaging. With the device of the invention each transducer can treat a respective area of the cornea. There is no need for displacement and focusing operations of the devices of the prior art. All necessary collagen shrinkage treatments in the cornea can be done simultaneously or subsequently without a repositioning of the device at an eye of the patient. Further, time consumption and facility consumption is reduced as both ocular imaging and thermal keratoplasty can be performed with a single device. Again no repositioning or changing of devices is necessary. Therefore, also the quality of treatment improves since the generated cornea images are fully aligned with the ultrasonic transducers for thermal keratoplasty. Since the device must not be repositioned or changed between ocular imaging and thermal keratoplasty a mismatch cannot occur. Ocular imaging can be done before and after the thermal keratoplasty treatments which enables to see the results immediately. If necessary, further thermal keratoplasty treatments can be done directly after the ocular imaging.

Therefore, the device according to the invention reduces the overall treatment time what makes it more comfortable for the patient. Finally, the device according to the invention also reduces the treatment cost for patients.

Preferably, all transducers of the plurality of transducers are capable of receiving ultrasound waves for ocular imaging. This enables a high resolution of the ocular images.

Preferably, the transducers are arranged in at least one ring array, wherein each ring array comprises a plurality of transducers in a circular consecutive manner, and wherein the transducers are at equal distance from each other in each ring array.

With this arrangement operation time is reduced because the transducers are distributed over the complete cornea areas to be treated, allowing treatment of several desired cornea areas at the same time. A ring array is the preferred arrangement of the transducers since this provides a high flexibility of use without the need for an angular repositioning of the device.

Preferably, the device comprises at least two concentric ring arrays of transducers. This particular arrangement allows the treatment of two concentric areas of the cornea, causing a belt effect generating a desired steepening of the central corneal region. At least two concentric ring arrays of transducers provide even a higher flexibility of use without the need for an angular or lateral repositioning of the device.

Preferably, each of the transducers comprises a plurality of transducer elements. Such transducer elements can be individually actuated in different phase to provide beam-forming of the ultrasound waves. This on the one hand enables the generation of the high power needed for the thermal keratoplasty and on the other hand allows for a precise beam focusing to a desired depth within the cornea without the need for additional focusing means like acoustic lenses. For applications without beam-forming the transducer elements can be commonly actuated in one common phase.

Preferably the transducer elements comprise a plurality of capacitive micro-machined ultrasonic transducer cells or at least one piezoelectric transducer sheet. Capacitive micromachined ultrasonic transducers (CMUTs) are a relatively new concept in the field of ultrasonic transducers. CMUTs are the transducers where the energy transduction is due to change in capacitance. As CMUTs are micromachined devices, it is easier to construct 2D arrays of transducers using this technology. This means large numbers of CMUTs could be included in a transducer array providing larger bandwidth compared to other transducer technologies. To achieve a high frequency operation using CMUTs is easier due to its smaller dimensions. Further, as it is usually built on silicon, the integration of electronics is easier for the CMUTs compared to other transducer technologies. The use of high frequency with a large bandwidth makes it a good choice to use CMUTs in a transducer in ocular imaging and thermal keratoplasty. In an alternative embodiment the each of the transducers comprises a plurality of piezoelectric transducer cells. Such piezoelectric transducer cells can also be used to generate ultrasonic waves from electrical signals. In other designs the transducer element can be made of a commonly known piezoelectric transducer sheet.

Preferably, the transducer elements are shaped in concentrical rings or a concentrical circle. Each transducer element preferably comprises of a ring or circle array of CMUT transducer cells or of at least one ring- or circle-shaped piezoelectric transducer sheet.

Preferably, the transducer cells within each transducer element are operated in the same phase or wherein the piezoelectric transducer sheet forming a transducer element is operated in one phase and, wherein the transducer elements are operated in separate phases for transmission beam focusing and the transducer elements are operated in the same phase for receiving ultrasound waves for ocular imaging. Focusing is simplified by using the plurality of micro-machined ultrasonic transducer cells (CMUT) or piezoelectric transduce cells that are driven in different phases. Thus the focusing depth can be adjusted very precisely and without any prior art lens systems. Further, only a single feeding means is needed for each ring array and a single phase shifting means for the device, what reduces system complexity and related costs. On the other hand for ocular imaging all rings of transducer cells operate in the same phase like a large single transducer. This enhances the sensitivity of the transducer.

Preferably, in one embodiment all of the transducer cells of one transducer are connected together to a single amplifier for receiving ultrasound waves. So, the transducer cells can act as a single reception transducer. This allows the use of a single amplifier, a moderate speed single analogue-digital-converter (ADC) and relatively simpler processing electronics. In other embodiments the transducer cells of one transducer are connected to individual amplifiers or amplifiers for groups of transducer cells to allow beam-forming during reception.

Preferably, the device further comprises a probe handle, wherein the transducers are integrated on a flat circular frontal side of the probe handle, a propagation funnel, wherein the propagation funnel is capable of holding a coupling fluid in between the transducers and the cornea, a valve for filling the propagation funnel with a coupling fluid, and a sapphire ring at the propagation funnel for contacting and sealing the device to the cornea. With this arrangement a compact and securely to handle device is provided. The coupling fluid may be a liquid or a gel in order to improve ultrasound beam propagation. Furthermore, the sapphire ring acts as a heat sink reducing the unwanted heating of the whole cornea and keeping the heating process highly localized. Further the sapphire ring acts as a suction ring to fix the device to the cornea. As it is almost air tight, the coupling fluid is securely held within the propagation funnel. By means of such a single system a complete cornea correction treatment can be performed without the need to change devices during treatment. Ocular imaging and thermal keratoplasty are done with one single system.

Particularly the above mentioned problem is also solved by a system for both ocular imaging and thermal keratoplasty, the system comprising a device as mentioned above, feeding means for feeding at least one transducer with electric energy for generating ultrasounds, and processing means for processing a signal, outputted by at least one of the transducers when ultrasounds are received, into cornea image information.

Particularly the above mentioned problem is also solved by a method for both ocular imaging and thermal keratoplasty, the method comprising the steps of:
a) providing a device comprising a plurality of ultrasonic transducers;
b) directing the transducers towards the cornea of an eye;
c) performing an image procedure by emitting ultrasounds with at least one of the transducers, receiving ultrasounds with at least one of the transducers, and processing a signal outputted by the at least one of the transducers receiving ultrasounds into cornea image information; and
d) performing a cornea heating procedure by emitting ultrasounds focused on the cornea with at least one of the transducers.

With the method of the invention each transducer may treat a particular area of the cornea. The need for displacement operations of a thermal keratoplasty device is eliminated. Further ocular imaging and thermal keratoplasty is done by the same device which reduces displacement errors, treatment time and treatment costs. Simultaneously the comfort for the patient is improved. Preferably all of the transducers are used for both emitting and receiving ultrasounds.

Preferably, at least one of the transducers is used for both emitting and receiving ultrasounds. The number of transducers can be reduced in this way. Therefore, overall cost and complexity of the device and method are reduced as well.

Preferably, the transducers are arranged in two concentric ring arrays, and wherein the focused ultrasounds are first emitted with a first ring array and subsequently with a second ring array, causing collagen shrinkage within the cornea. This subsequent treatment will cause a belt like effect inducing a desired steepening of the central corneal region.

Preferably, the method further comprises the step of using the cornea image information to calculate the focusing depth of the ultrasound waves transmitted by the at least one transducer. It is therefore not necessary to use another device and method to calculate the focusing depth before the providing the device. This saves time and costs. Furthermore, precision is increased because the device stays in the same position for heating the cornea after the ocular imaging and focusing depth calculation.

Preferably, each of the transducers comprises a plurality of capacitive micro-machined ultrasonic transducer cells or piezoelectric transducer sheets and some of the capacitive micro-machined ultrasonic transducer cells or piezoelectric transducer sheets are operated in separate phases for beam focusing.

Preferably, an additional imaging procedure can be performed after a first thermal keratoplasty for analyzing the depth of the coagulation effect and the occurred shrinkage after the cornea heating procedure. So it can be decided whether the treatment was successful or whether additional thermal keratoplasty steps of the method need to be performed.

Further preferred embodiments are described in the dependent claims.

### 4. BRIEF DESCRIPTION OF THE DRAWINGS

The features and advantages of the present invention will become more apparent from the detailed description set forth below when taken in conjunction with the drawings in which like reference characters identify corresponding elements in the different drawings.

FIG. 1 shows a top frontal view of a preferred embodiment of a device according to the invention, wherein ultrasonic transducers are arranged in a circular consecutive manner.

FIG. 2 shows a sectional side view along line A-A in FIG. 1 of the preferred embodiment of the device of FIG. 1 applied to an eye.

FIG. 3 shows a three dimensional side view of the preferred embodiment of the device of FIG. 1 applied to an eye.

FIG. 4 shows a top frontal view of a preferred embodiment of an ultrasound transducer with a plurality of CMUT transducer cells or piezoelectric transducer cells used in a device according to the invention.

### 5. DETAILED DESCRIPTION OF PREFERRED EMBODIMENTS

In the following preferred embodiments of the invention are described with reference to the figures.

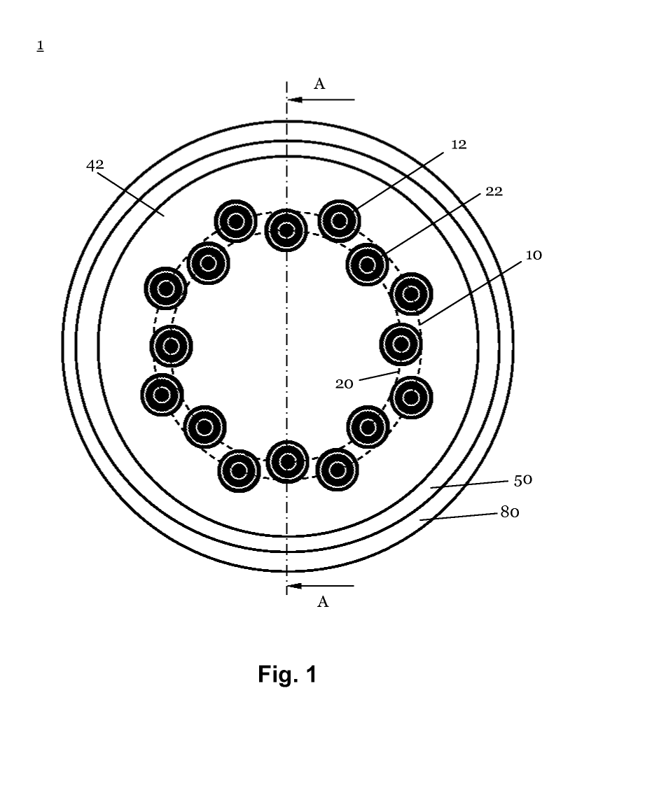

FIG. 1 shows a top frontal view of a preferred embodiment of a device 1 for both ocular imaging and thermal keratoplasty. With the device 1 both ocular Imaging (for example A-scan ultrasonography) by high frequency ultrasound and thermal keratoplasty by High Intensity Focused Ultrasound (HIFU) can be performed with the same device subsequently and directly at the eye of a patient (in vivo).

As shown in the top-frontal view of FIG. 1 the device 1 comprises two ring arrays 10, 20 of a number of ultrasound transducers 12, 22. The transducers 12, 22 are arranged on the ring arrays 10, 20 in a circular consecutive manner in two concentric ring arrays 10, 20. In the shown exemplary embodiment the diameter of the inner ring array 20 is 6 mm and the diameter of the outer ring array 10 is 7.2 mm. In other embodiments also other diameters or shapes of arrays are possible. The diameter of the ring arrays 10, 20 can be chosen according to the needs of the desired therapy. It is preferred, that the operator can select different devices with different ring array diameters or other distribution of the transducers 12, 22 on the device 1. In the shown embodiment eight transducers 22 are arranged in the inner ring array 20 and eight transducers 12 are arranged in the outer ring array 10. Of course, other numbers of transducers are possible.

Preferably, the transducers 12, 22 are at equal distance from each other in each ring array 10, 20, meaning, the transducers 22 arranged on the 6 mm circle 20 are at equal distance from each other and the same applies for the transducers 12 arranged in the 7.2 mm circle 10. These particular arrangements of transducers 12, 22 are intended to accommodate equally sized transducers 12, 22 on a front side 42 of a probe handle 40.

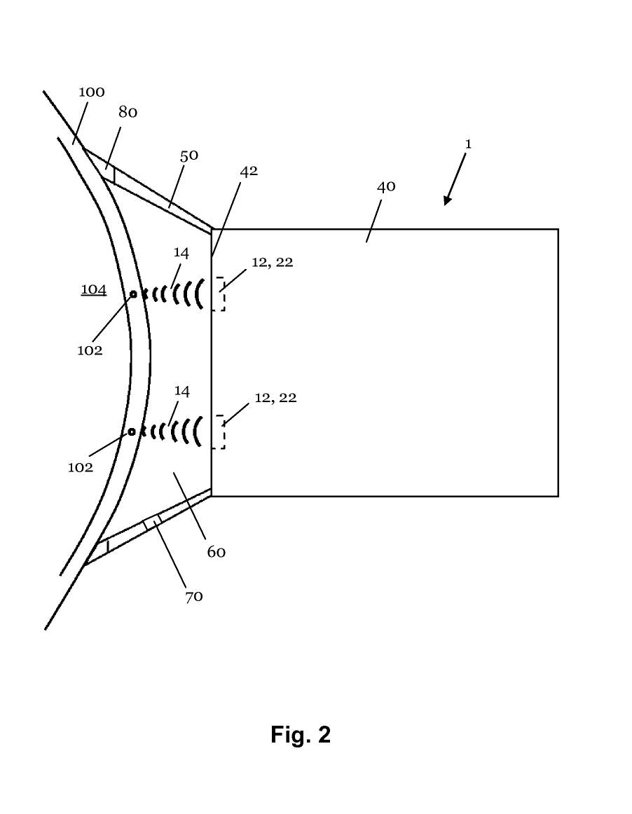

As it is illustrated in FIG. 2 each of the transducers 12, 22 is intended to fire ultrasound beams 14 focused on the stromal layer (within the range of 100-500 μm thickness seeing from the top) of the cornea 100 of an eye 104. The focused ultrasound beams 14 cause local collagen shrinkage in focal spots 102. This collagen shrinkage in and around the focal spots 102 will cause a belt like effect causing the steepening of the central corneal region what in the end generates the desired optical correction of the eye 100. Preferably in exemplary embodiments, the diameter of the focal spots 102 is about 30 μm to 80 μm, preferably 50 μm.

The optimal temperature needed for the collagen shrinkage in human cornea 100 is restricted to about 80 degree centigrade. Beyond 80 degree temperature corneal stroma is subject to thermal damage. Optionally, if required, the threshold temperature needed for stromal collagen shrinkage can be reduced by the usage of reagents like lysozyme. Preferably, the focal spots 102 lesions are made at equal distance from each other. Such a distribution leads to the best optical results.

Referring to FIG. 2, the device 1 comprises an elongated probe handle 40, wherein the transducers 12, 22 are arranged on a flat circular front side 42 thereof. In this embodiment the probe handle 40 is an elongated cylindrical structure but any suitable other form may be possible.

The probe handle 20 is attached to a hollow propagation funnel 50 broadening gradually as it protrudes away from the forward edge of the probe handle 40. The propagation funnel 50 is preferably air tight attached to the probe handle 40 and is capable of holding a coupling fluid 60 in between the transducers 12, 22 and the cornea 100. To this end the propagation funnel 50 is fill-able and refillable with coupling fluid 60 which may be a liquid or gel. The coupling fluid 60 improves ultrasound beam propagation. It can be filled into the propagation funnel 50 through a sealable valve 70 located on the side of the propagation funnel 50. The length of the propagation funnel 50 depends on the calculated path length of the transmitted and received ultrasound beams 14. For 1 mm diameter transducers 12, 22 the focusing distance is approximately 2 mm.

The front side the propagation funnel 50 terminates into a sapphire ring 80 that works as a corneal suction ring. As the suction ring 80 is almost air tight, the coupling fluid 60 does not leak from the sides out of the suction ring 80. Furthermore, the sapphire suction ring 80 acts as a heat sink and reduces an unwanted heating of the whole cornea 100. Thus, the sapphire suction ring 80 keeps the heating process highly localized. Preferably, the inner diameter of the sapphire suction ring 80 is approximately the same as the diameter of a typical cornea 100. An inner diameter of 11.5 mm for the sapphire suction ring 80 is preferred. In the exemplary embodiment the outer diameter of the sapphire suction ring 80 is 13.5 mm.

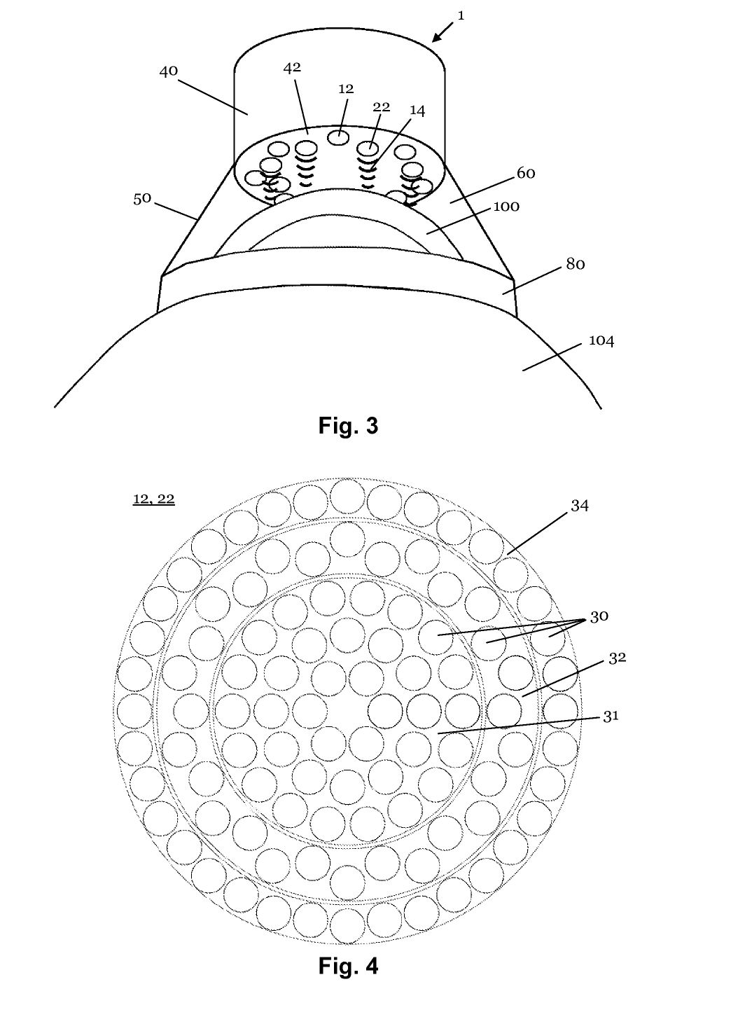

FIG. 3 shows the use of the device of FIGS. 1 and 2 on an eye 104 for ocular imaging and thermal keratoplasty. The propagation funnel 50 may be transparent to control the correct positioning of the device 1 on the cornea 100.

As shown in FIG. 4 each transducer ultrasound 12, 22 comprises a plurality of Capacitive Micro-machined Ultrasound Transducer cells (CMUT cells) 30. Some of the CMUT cells 30 are grouped in two concentrical ring shaped transducer elements 32, 34 and some of the CMUT cells 30 are grouped in a circular transducer element 31. Preferably all CMUT cells 30 within one transducer element 31, 32, 34 are commonly actuated so that they are in-phase to each other.

In a different embodiment the transducer elements 31, 32, 34 can be made of commonly known piezoelectric transducer sheets. In this embodiment the piezoelectric transducer sheets would be ring shaped corresponding to the concentrical rings of transducer elements 32, 34 and would be circular shaped corresponding to the circular transducer element 31 on the central area of the transducer 12, 22.

In FIG. 4 for ease of display the transducer cells 30 are shown very large. In reality CMUT cells 30 are very small and can have a diameter of about 10-100 μm. Therefore, in reality each shown CMUT cell 30 may comprise an array of a plurality CMUT cells. Such an array may comprise hundreds of CMUT cells. Each CMUT cell 30 is designed and fabricated for broad band and high frequency ultrasound beam transmission and reception. Preferably, the whole area of each ring array 34, 32 and the inner area 31 is to be covered with as many CMUT cells 30 as possible, such that the HIFU beam accumulation can be maximum, producing the maximum available heat during collagen shrinkage.

Although, CMUT cells 30 are preferred, alternatively, the transducers 12, 20 may comprise conventional PVDF transducer elements or may be constructed of other materials, including crystalline quartz, or piezoelectric materials, such as zirconium titanite, lithium noibide, lead zirconate titanate (PZT) or a lead zirconium.

For ultrasound beam transmission the transducer cells 30 of on one ring 34, for example CMUT transducer cells 30, can be operated with a different phase as the transducer cells 30 of the other ring 32, enabling focusing or beam forming of the ultrasounds beam 14. More than two rings or ring shaped transducer elements 32, 34 of transducer cells 30 are possible on one transducer 12, 22. Further it is possible to arrange the transducer cells 30 in different arrangement on one transducer 12, 22, however the ring arrangement is preferred in view of focusing or beam forming of the ultrasound beams 14.

However, beamforming is not always necessary for reception of ultrasound waves. Implementing beamforming capability in the receiving mode would require separate amplifiers for every transducer cell 30 or for every ring array 32, 34 of transducer cells 30 and complex digital electronics for processing. Therefore, it is preferred that in the receiving mode for acoustic imaging the transducer cells 30 of one transducer work 12, 22 together as they would be a simple piston transducer. For instance, the transducer cells 30 of each ring array 32, 34 can be operated together so that the entire ring array acts as a simple piston transducer. The first ring array 32 and the second ring array 34 can then be operated in the same phase. Then a single amplifier, a moderate speed single analog-to-digital converter and relatively simpler processing electronics would be enough for the device. The simpler electronics leads to less design oriented costs compared to the costs of the laser devices in LTK and Opti-K.

The driving electronics for the ultrasound transducer system can be integrated within the probe handle 40. The driving electronics comprises a single integrated circuit or sub-blocks of the electronics and includes pulse/signal drivers and/or transmit/receive switches and/or protection circuitry and/or preamplifiers and/or analog-to-digital converters. Due to their physical structure, CMUT cells 30 require relatively high drive voltages for the generation of the acoustic field. Hence in an exemplary embodiment a high-voltage (50 V) CMOS technology can be used in the design of the drive electronics.

The frequency of operation and the transducer 12, 22 diameters define the spot-size of the HIFU lesion. For an example, for a 50 μm spot size the transducer 12, 22 operation frequency is preferably 30 MHz and the transducer 12, 22 diameter is about 1 mm. Both A-scan and HIFU heating are feasible around such a frequency range.

In the following a preferred treatment procedure for both ocular imaging and thermal keratoplasty which may use the above described device is described in detail.

One drop of anesthetic is instilled into the eye 104 under treatment. Optionally if required, a drop of lysozyme is also instilled along with the anesthetic, in case the threshold shrinkage temperature is to be reduced. A lid speculum holds the eyelid. The eye 104 is irrigated to prevent dryness.

Then the sapphire suction ring 80 is set in contact with the cornea 100. Then a coupling fluid 60 is poured into the propagation funnel 50 through the valve 70 and subsequently the valve 70 is closed.

Then the image procedure is performed. One or more transducers 12, 22 fire high frequency beams 14 to the cornea 100 and image the cornea 10 in the depth direction. This imaging process gives an idea of the depth configuration of the cornea 100 under treatment. The images are subsequently assessed on a computer screen. The images tell at what depth the HIFU beams 14 are to be focused. The depth can be deduced by the operator from the image or calculated by a computer program.

The HIFU beams may be focused by shifting the drive voltage phases between CMUT cells 30 or piezoelectric transducer cells 30 on the ring arrays 30, 32 of each transducer 12, 22. This can be done manually or automatically, for instance with a computer program. In the same way the temperature needed and the time that the HIFU beams 14 should be emitted is calculated. Then focused HIFU beams 14 are fired, causing collagen shrinkage in the respective focal spots 102 within the cornea 100.

The focused HIFU beams 14 may be first fired from some or all transducers 12 of the first ring array 10 and then from some or all transducers 22 of the second ring array 20. This may cause a belt like effect causing the steepening of the central corneal region. The required HIFU beams 14 are applied for a calculated amount of time to produce the desired temperature and spot 102 size and are then switched off.

Subsequently a further imaging procedure is performed for analyzing the in depth coagulation effect and the occurred shrinkage. So the operator can decided whether the treatment was successful or whether the thermal keratoplasty steps must be repeated.

The whole thermal keratoplasty and ocular imaging procedure may be controlled automatically by the system, for instance by means of a computer program. This includes also repeating the procedure several times until the desired result is achieved. Of course in the procedure may also be controlled by an operator, taking the required decisions.

As herein disclosed, ocular imaging and thermal keratoplasty are performed by the same device 1. Ring arrays of CMUT cells 30 or piezoelectric transducer cells 30 are proposed to perform both A-scan ultrasonography and HIFU guided thermal keratoplasty in vivo. This reduces the time consumption and facility consumption as dual jobs are performed by a single system integrated in single packaging. This further reduces treatment cost for patients as well. With laser keratoplasty, for example by LTK or Opt-K, this dual modality cannot be established and hence treatment cost are higher than with a device according to the invention.

What has been described above includes examples of one or more embodiments. It is, of course, not possible to describe every conceivable combination, or permutation, of components and/or methodologies for purposes of describing the aforementioned embodiments. However, one of ordinary skill in the art will recognize that many further combinations and permutations of various embodiments are possible within the general inventive concept derivable from a direct and objective reading of the present disclosure. Accordingly, it is intended to embrace all such alterations, modifications and variations that fall within scope of the appended claims.

### LIST OF REFERENCE SIGNS

1 device for thermal keratoplasty
10, 20 array of transducers
12, 22 ultrasonic transducers
14 ultrasound waves
30 capacitive micro-machined ultrasonic transducer cell
31 circular shaped transducer element
32, 34 ring shaped transducer elements
40 probe handle
42 frontal side of probe handle
50 propagation funnel
60 coupling fluid
70 valve
80 sapphire ring
100 cornea
104 eye

1. A thermal keratoplasty device comprising:
a) a plurality of ultrasonic transducers for emitting ultrasound waves,
b) at least one of the transducers is focusing its ultrasound waves on a corresponding area of the cornea in order to heat these area and cause collagen shrinkage;
c) at least one of the transducers is capable of receiving ultrasound waves for ocular imaging; and
d) a propagation funnel, wherein the propagation funnel is capable of holding a coupling fluid in between the transducers and the cornea.

2. The device according claim 1, wherein all transducers of the plurality of transducers are capable of receiving ultrasound waves for ocular imaging.

3. The device according to claim 1, wherein the transducers are arranged in at least one ring array, wherein each ring array comprise a plurality of transducers in a circular consecutive manner, wherein the transducers are at equal distance from each other in each ring array.

4. The device according to claim 3, comprising at least two concentric ring arrays of transducers.

5. The device according to claim 1, wherein each of the transducers comprises a plurality of transducer elements.

6. The device according claim 5 wherein the transducer elements comprise a plurality of capacitive micro-machined ultrasonic transducer cells or at least one piezoelectric transducer sheet.

7. The device according claim 6, wherein the transducer elements are shaped in concentrical rings or a concentrical circle.

8. The device according to claim 6, wherein the transducer cells within each transducer element operates in the same phase or wherein the piezoelectric transducer sheet forming a transducer element operates in one phase; and wherein
a) the transducer elements operate in separate phases for transmission beam focusing; and
b) the transducer elements operate in the same phase for receiving ultrasound waves for ocular imaging.

9. The device according to claim 1, further comprising:
a) a probe handle, wherein the transducers are integrated on a flat circular frontal side of the probe handle; and
b) a valve for filling the propagation funnel with a coupling fluid; and
c) a sapphire ring at the propagation funnel for contacting and sealing the device to the cornea.

10. A system for both ocular imaging and thermal keratoplasty, the system comprising:
a) the thermal keratoplasty device of claim 1;
b) feeding means for feeding at least one transducer with electric energy for generating ultrasounds; and
c) processing means for processing a signal, outputted by at least one of the transducers when ultrasounds are received, into cornea image information.

11-15. (canceled)
